(19) United States
(12) Patent Application Publication
(10) Pub. No.: US 2021/0145472 A1
(43) Pub. Date: May 20, 2021

(54) HANDLE CONFIGURATIONS FOR SURGICAL INSTRUMENTS

(71) Applicant: Covidien LP, Mansfield, MA (US)

(72) Inventor: Hung Lieu, Westminster, CO (US)

(21) Appl. No.: 17/028,446

(22) Filed: Sep. 22, 2020

Related U.S. Application Data
(60) Provisional application No. 62/937,910, filed on Nov. 20, 2019.

(51) Int. Cl. A61B 17/32

(52) U.S. Cl. CPC A61B 17/320092; A61B 2017/2925

(57) ABSTRACT

A surgical instrument includes a handle assembly including a body portion and a handle portion. The handle portion is operably coupled to and extends from the body portion. The handle portion includes a fixed handle and a trigger selectively actuatable relative to the fixed handle. The fixed handle defines a first axis therethrough. An elongated assembly extends distally from the handle assembly. An end-effector is supported at a distal end portion of the elongated assembly. The handle portion of the handle assembly is rotatable about the first axis with respect to the body portion of the handle assembly.

### CROSS-REFERENCE TO RELATED APPLICATIONS

This application claims the benefit of and priority to U.S. Provisional Patent Application Ser. No. 62/937,910 filed Nov. 20, 2019, the entire disclosure of which is incorporated by reference herein.

### BACKGROUND

### Technical Field

The present disclosure relates generally to shaft-based surgical instruments and, more particularly, the present disclosure relates to handle configurations for surgical instruments.

### Discussion of Related Art

Ultrasonic surgical instruments utilize ultrasonic energy, i.e., ultrasonic vibrations, to treat tissue. More specifically, ultrasonic surgical instruments utilize mechanical vibration energy transmitted at ultrasonic frequencies to coagulate, cauterize, fuse, seal, cut, desiccate, and/or fulgurate tissue to affect hemostasis.

Shaft-based ultrasonic surgical instruments transmit ultrasonic energy produced by a generator and transducer assembly along a waveguide to an end effector assembly that is spaced-apart from the generator and transducer assembly. Thus, the end effector assembly may be positioned within an internal surgical site, e.g., inserted through a cannula assembly, while the generator and transducer assembly remains externally disposed, e.g., in order to perform so-called “minimally-invasive” surgical procedures.

### SUMMARY

In accordance with an aspect of the present disclosure, a surgical instrument includes a handle assembly including a body portion and a handle portion. The handle portion is operably coupled to and extends from the body portion. The handle portion includes a fixed handle and a trigger selectively actuatable relative to the fixed handle. The fixed handle defines a first axis therethrough. An elongated assembly extends distally from the handle assembly. An end-effector is supported at a distal end portion of the elongated assembly. The handle portion of the handle assembly is rotatable about the first axis with respect to the body portion of the handle assembly.

In some aspects, the body portion and the handle portion define a pistol-grip configuration.

In some aspects, the fixed handle and the trigger are configured to rotate in conjunction with one another about the first axis with respect to the body portion.

In some aspects, the surgical instrument includes an ultrasonic transducer and generator assembly (TAG). The TAG is configured for releasable engagement with the body portion of the handle assembly.

In some aspects, the handle portion is configured to rotate 360 degrees about the first axis with respect to the body portion. A locking sleeve may secure the handle portion in a fixed rotational position relative to the body portion.

In some aspects, the trigger is selectively actuatable to actuate the end effector to clamp tissue.

In some aspects, the fixed handle of the handle portion includes a battery compartment formed therein. The battery compartment may include a door positioned at a bottom region of the fixed handle.

In some aspects, a rotation knob may be disposed on the body portion of the handle assembly. The rotation knob rotates the elongated assembly with respect to the handle assembly.

In accordance with an aspect of the present disclosure, a surgical instrument includes a handle assembly defining a pistol-grip configuration including a body portion. A fixed handle extends from the body portion. A trigger extends from the body portion adjacent the fixed handle. The fixed handle defines a vertical axis therethrough. An elongated assembly extends distally from the body portion of the handle assembly. An end-effector is supported at a distal end portion of the elongated assembly. The trigger and the fixed handle are rotatable about the vertical axis with respect to the body portion.

In some aspects, the fixed handle and the trigger extending therefrom rotate in conjunction with one another about the vertical axis with respect to the pistol-grip body portion.

### BRIEF DESCRIPTION OF THE DRAWINGS

The accompanying drawings, which are incorporated in and constitute a part of this specification, illustrate aspects and features of the present disclosure and, together with the detailed description below, serve to further explain the present disclosure, in which:

FIG. 1 is a perspective view of an ultrasonic surgical instrument in accordance with the present disclosure;

FIG. 2A is a perspective view of the ultrasonic surgical instrument of FIG. 1, wherein a transducer and generator assembly (“TAG”) is disengaged from a handle assembly of the ultrasonic surgical instrument;

FIGS. 2B-1 and 2B-2 illustrate exemplary attachment mechanisms configured for use with the ultrasonic surgical instrument of FIG. 1;

FIGS. 3A and 3B are side views of another handle assembly configured for use with the ultrasonic surgical instrument of FIG. 1, wherein the handle assembly includes a slidable handle attached to a body portion of the ultrasonic surgical instrument; and

FIGS. 4A and 4B are side views of another handle assembly configured for use with the ultrasonic surgical instrument of FIG. 1, wherein the handle assembly includes a handle rotatably attached to a body portion of the ultrasonic surgical instrument of FIG. 1.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about +or−10 degrees from true parallel and true perpendicular.

“About” or “approximately” or “substantially” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings.

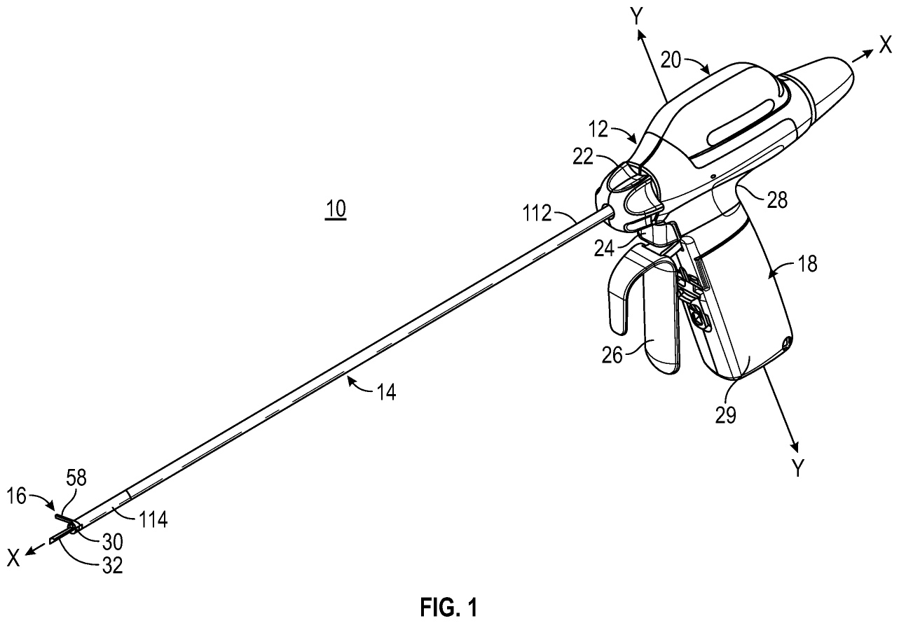

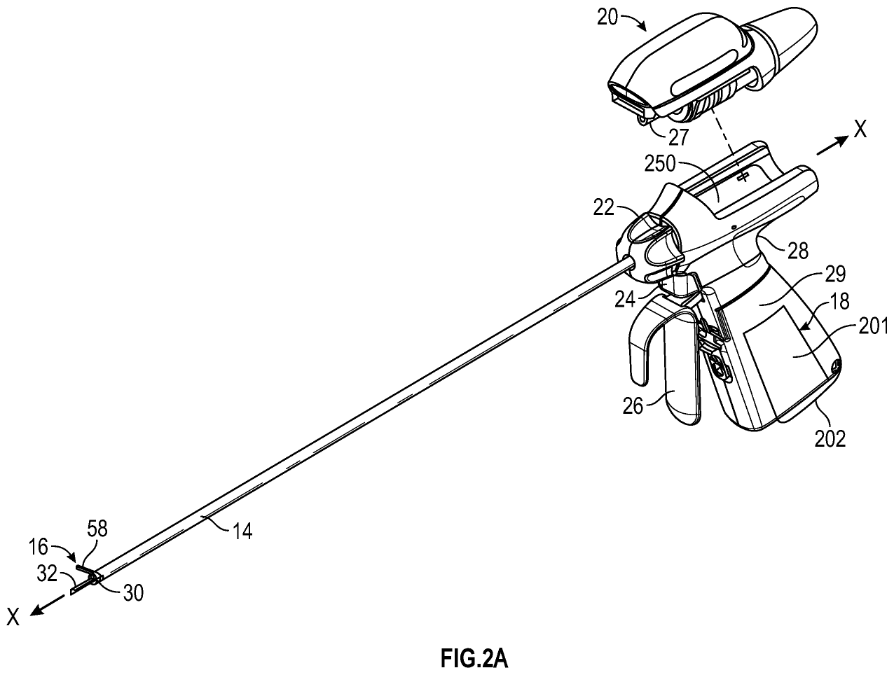

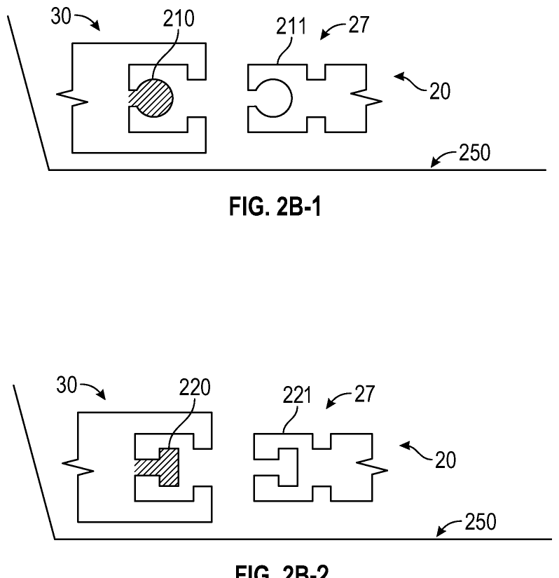

Referring to FIGS. 1 and 2A, one embodiment of a surgical instrument exemplifying the features of the present disclosure is shown generally identified as ultrasonic surgical instrument 10. Instrument 10 includes a handle assembly 12, a shaft 14, and an end effector 16. Handle assembly 12 supports a battery assembly 18 and an ultrasonic transducer and generator assembly (“TAG”) 20, and includes a rotatable knob 22, an activation button 24, and a clamp trigger 26. Battery assembly 18 and TAG 20 are each releasably secured to a body portion 28 of handle assembly 12, and are removable from body portion 28 to facilitate disposal of ultrasonic instrument 10, with the exception of battery assembly 18 and TAG 20, or to facilitate sterilization of some or all of the components of instrument 10. As illustrated in FIG. 1, battery assembly 18 defines a fixed handle portion of handle assembly 12 when engaged thereto. Alternatively, as illustrated in FIG. 2A, battery assembly 18 may be insertable into a compartment 201 defined within a fixed handle portion 29 of handle assembly 12 and enclosed therein via a movable door 202 to enable aseptic transfer and receipt of battery assembly 18 within fixed handle portion 29 of handle assembly 12.

Body portion 28 of handle assembly 12 defines a recess 250 therein that is configured to receive TAG 20. TAG 20 is removably engagable within recess 250 of body portion 28, e.g., via snap-fit engagement. When engaged within recess 250, TAG 20 is electrically coupled to battery assembly 18, permitting battery assembly 18 to supply power to TAG 20.

As an alternative to a handheld, cordless device, instrument 10 may alternatively be configured to connect to a tabletop generator (not shown). In such configurations, the transducer of TAG 20 is configured to releasably engage handle assembly 12 and waveguide 30 as detailed herein and is connected to the generator (which itself is connected to a power source, e.g., a wall outlet) via a surgical cable (not shown). Thus, neither the generator nor battery assembly is required for engagement on or within instrument 10.

Shaft 14 defines a longitudinal axis “X-X” and includes the end effector 16 extending distally therefrom. The end effector 16 includes a blade 32 extending from shaft 14 and a clamp arm 58 operably coupled to shaft 14. A waveguide 30 extends through shaft 14 and includes a proximal end configured to engage the TAG 20 and a distal end that connects with or defines the blade 32. The waveguide 30 is configured to transmit ultrasonic energy produced by TAG 20 to blade 32 for treating tissue.

End effector 16 includes a clamp member 58 that is pivotable relative to blade 32 between an open position and a clamping position for grasping tissue therebetween. The clamp member 58 is moved from the open position to the clamping position in response to actuation of the clamp trigger 26 of handle assembly 12.

Continuing with reference to FIGS. 1 and 2A, clamp trigger 26 of handle assembly 12 cooperates with a drive assembly (not shown) to transition clamp member 58 from the open position to the clamping position upon depression, or squeezing of clamp trigger 26 towards battery assembly 18. Activation button 24 extends distally from handle assembly 12 adjacent clamp trigger 26. Activation button 24 is configured to selectively activate battery assembly 18 and TAG 20 to supply ultrasonic energy to blade 32 in two power settings, e.g., a high power mode and a low power mode, although other configurations are also contemplated. Rotatable knob 22 is rotatable in either direction about longitudinal axis “X-X” to rotate end effector 16 about longitudinal axis “X-X” to better position end effector 16 for clamping and treating tissue.

In preparation for use, battery assembly 18 is engaged to handle assembly 12 and TAG 20 is engaged within recess 250 of body portion 28 of handle assembly 12. TAG 20 is also engaged to the waveguide 30 and is electrically coupled to battery assembly 18, either simultaneously with the engagement of TAG 20 within recess 250 of body portion 28, sequentially therewith, or independently thereof.

With instrument 10 in the assembled condition, instrument 10 is advanced into the surgical site and manipulated such that end effector 16 is positioned with tissue to be treated disposed between clamp member 58 and blade 32 thereof. Thereafter, clamp trigger 26 is depressed, or squeezed towards battery assembly 18 to transition clamp member 58 to the clamping position to clamp tissue between clamp member 58 and blade 32. Blade 32 may then be activated, e.g., activation button 24 may be depressed, to supply ultrasonic energy from TAG 20, along the waveguide 30, to blade 32. Ultimately, the ultrasonic energy provided at blade 32 is used to seal, dissect, or otherwise treat tissue clamped between clamp member 58 and blade 32. As described in more detail below, in embodiments, the handle portion of the handle assembly 12 may be manipulated, e.g., rotated and/or slid, to adjust an angle between a user's wrist and the body portion 28 and/or to adjust the spacing between battery assembly 18 and clamp trigger 26. Accordingly, an amount of strain to user's hand is reduced, thus increasing the comfortability of using the instrument 10, while improving the ability of a user to manipulate a position of the end effector 16.

As can be appreciated, in order to properly control the ultrasonic energy provided at blade 32, to ensure proper functionality, and to maintain the engagement between waveguide 30 and TAG 20 during use, waveguide 30 and ultrasonic horn 27 of TAG 20 should be properly and sufficiently secured to one another (e.g., to ensure proper transmission of the standing ultrasonic wave from the ultrasonic transducer of TAG 20 to and along waveguide 30 via ultrasonic horn 27). Further, since waveguide 30 and/or TAG 20 are releasably engagable with one another (and with handle assembly 12), an engagement configuration should be consistently repeatable for each subsequent engagement of TAG 20 and waveguide 30 to help ensure an effective engagement each time TAG 20 and waveguide 30 are engaged to one another. For example, TAG 20 is removably engagable within recess 250 of body portion 28, via snap-fit engagement or other suitable releasable locking engagements, e.g., latching.

Referring to FIGS. 2B-1 and 2B-2, and initially to FIG. 2B-1, with respect to the engagement of the proximal end of waveguide 30 with ultrasonic horn 27 of TAG 20, a slot 221 and key 220 engagement configuration may be employed to removably engage the proximal end of waveguide 30 with ultrasonic horn 27 of TAG 20 within recess 250 under sufficient compression to enable the efficient transmission of ultrasonic energy from horn ultrasonic horn 27 to waveguide 30. This may be accomplished via transverse sliding of ultrasonic horn 27 relative to waveguide 30, e.g., laterally or vertically, such that the complementary slot 221 and key 220 are inter-fit and engaged with one another, e.g., in a snap-fit manner. Although a slot 221 and key 220 are illustrated as disposed on waveguide 30 and ultrasonic horn 27, respectively, this configuration may be reversed.

With additional reference to FIG. 2B-1, alternatively, a trailer hitch engagement configuration including a male end 210 and a female 211 end may be employed to removably engage waveguide 30 and ultrasonic horn 27 with one another within recess 250, similarly as detailed above. This configuration may also be reversed.

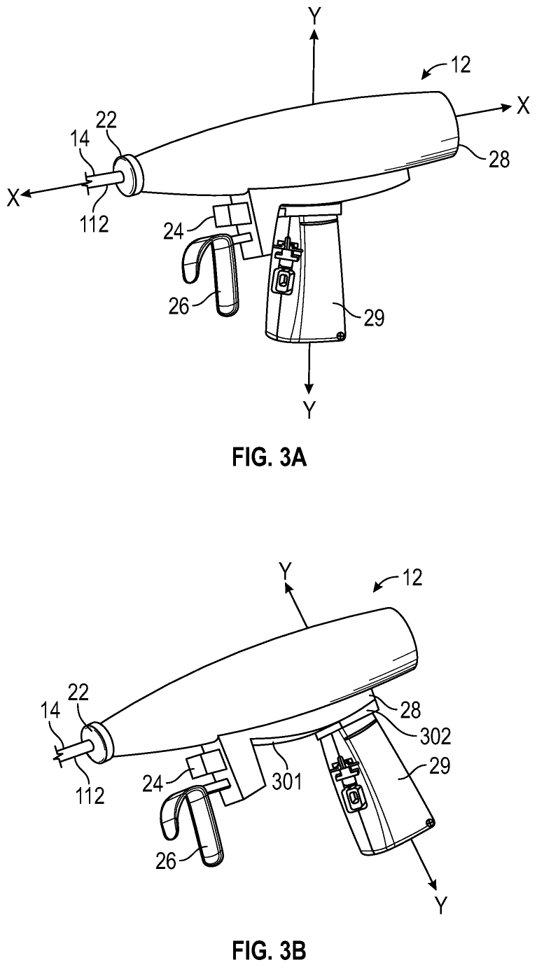

Referring to FIGS. 3A and 3B, according to embodiments of the present disclosure, fixed handle 29 of handle assembly 12 (whether formed via battery assembly 18 (FIG. 1), retaining battery assembly 18 therein (FIG. 2A), or excluding a battery assembly altogether) is adjustable relative to body portion 28 of handle assembly 12. The fixed handle 29 and the body portion 28 may cooperate to define a pistol grip configuration. Fixed handle 29 may slide along body portion 28 in a generally longitudinal direction relative to longitudinal axis “X-X” to be positioned at varying distances from clamp trigger 26, although fixed handle 29 may, in embodiments, be configured to move along an arcuate path to increase ergonomics. Body portion 28 may include a track 301 along which the fixed handle 29 slides. The fixed handle 29 may include a stop member 302 to releasably secure, e.g., lock, the fixed handle 29 at a desired position along track 301. The sliding configuration of fixed handle 29 allows the handle assembly 12 to comfortably accommodate hands of varying sizes while allowing access to clamp trigger 26 and activation button 24.

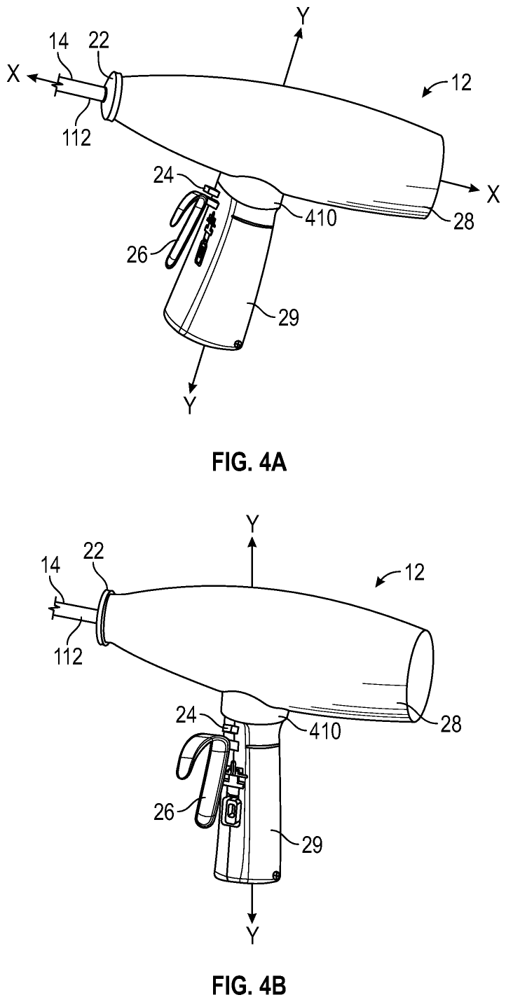

Referring to FIGS. 4A and 4B, in embodiments, instrument 10 includes a handle assembly 12 including a body portion 28 and a handle portion (e.g., including clamp trigger 26, activation button 24, and fixed handle 29). The handle portion is operably coupled to and extends from the body portion 28. The handle portion includes clamp trigger 26 which is selectively actuatable relative to the fixed handle 29. The fixed handle 29 defines an axis (e.g., axis “Y-Y”) therethrough. The fixed handle 29, as detailed above, may be formed via battery assembly 18 (FIG. 1), retain battery assembly 18 therein (FIG. 2A), or exclude a battery assembly altogether. Axis “Y-Y” may be substantially perpendicular or orthogonal to axis “X-X”. Axis “Y-Y” may be a substantially vertical axis. Similarly as detailed above, shaft 14 extends distally from the handle assembly 12 to support end-effector 16 (FIG. 1) at a distal end thereof. The handle portion of the handle assembly 12 is rotatable about the “Y-Y” axis with respect to the body portion 28 of the handle assembly 12.

The fixed handle 29 and the clamp trigger 26 (and, in embodiments, the activation button 24) may rotate in conjunction with one another about a vertical axis (e.g., the “Y-Y” axis) with respect to the body portion 28. Rotation of the fixed handle 29 relative to the body portion 28 limits wrist strain of a user and allows for increased accuracy in positioning the end effector 16 (FIG. 1).

In embodiments of the present disclosure, the body portion 28 and the handle portion cooperate to define a pistol-grip configuration.

The handle portion (e.g., including fixed handle 29, clamp trigger 26, and activation) is configured to rotate up to or continuously through 360 degrees about the axis “Y-Y” with respect to the body portion 28 (i.e., the handle portion may be rotated more or less than 360 degrees). A locking sleeve 410 may releasably secure the handle portion in a fixed rotational position relative to the body portion 28. For example, the locking sleeve 410 may engage a rotation detent. Thus, the locking sleeve 410 prevents undesired rotation of the handle portion relative to the body portion 28.

In embodiments, with reference to FIGS. 3A-4B, the sliding adjustability of fixed handle 29 relative to clamp trigger 26 (and, in embodiments, activation button 24) may be combined with the rotation adjustability of the handle portion (e.g., clamp trigger 26, activation button 24, and fixed handle 29) relative to body portion 28 such that the position of fixed handle 29 may selectively be slid relative to clamp trigger 26 and activation button 24 and/or such that fixed handle 29, clamp trigger 26, and activation button 24 may be selectively rotated relative to body portion 28, thereby providing a desired ergonomic configuration.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument, comprising:
a handle assembly including a body portion and a handle portion operably coupled to and extending from the body portion, the handle portion including a fixed handle and a trigger selectively actuatable relative to the fixed handle, the fixed handle defining a first axis therethrough;
an elongated assembly extending distally from the handle assembly; and
an end-effector supported at a distal end portion of the elongated assembly,
wherein the handle portion of the handle assembly is rotatable about the first axis with respect to the body portion of the handle assembly.

2. The surgical instrument of claim 1, wherein the body portion and the handle portion define a pistol-grip configuration.

3. The surgical instrument of claim 1, wherein the fixed handle and the trigger are configured to rotate in conjunction with one another about the first axis with respect to the body portion.

4. The surgical instrument of claim 1, further comprising an ultrasonic transducer and generator assembly (TAG).

5. The surgical instrument of claim 4, wherein the TAG is configured for releasable engagement with the body portion of the handle assembly.

6. The surgical instrument of claim 1, wherein the handle portion is configured to rotate 360 degrees about the first axis with respect to the body portion.

7. The surgical instrument of claim 1, further including a rotation lock configured to secure the handle portion in a fixed rotational position relative to the body portion.

8. The surgical instrument of claim 1, wherein the trigger is selectively actuatable to actuate the end effector to thereby clamp tissue.

9. The surgical instrument of claim 1, wherein the fixed handle of the handle portion includes a battery compartment formed therein.

10. The surgical instrument of claim 9, wherein the battery compartment includes a door positioned at a bottom region of the fixed handle.

11. The surgical instrument of claim 1, further including a rotation knob disposed on the body portion of the handle assembly and configured to rotate the elongated assembly with respect to the handle assembly.

12. A surgical instrument, comprising:
a handle assembly defining a pistol-grip configuration including a body portion, a fixed handle extending from the body portion, and a trigger extending from the body portion adjacent the fixed handle, the fixed handle defining a vertical axis therethrough;
an elongated assembly extending distally from the body portion of the handle assembly; and
an end-effector supported at a distal end portion of the elongated assembly,
wherein the trigger and the fixed handle are rotatable about the vertical axis with respect to the body portion.

13. The surgical instrument of claim 12, wherein the fixed handle and the trigger extending therefrom are configured to rotate in conjunction with one another about the vertical axis with respect to the pistol-grip body portion.

14. The surgical instrument of claim 12, further comprising an ultrasonic transducer and generator assembly (TAG).

15. The surgical instrument of claim 14, wherein the TAG is configured for releasable engagement with the body portion of the handle assembly.

16. The surgical instrument of claim 12, wherein the handle portion is configured to rotate 360 degrees about the vertical axis with respect to the pistol-grip body portion.

17. The surgical instrument of claim 12, further including a locking sleeve configured to secure the handle portion in a fixed rotational position relative to the body portion.

18. The surgical instrument of claim 12, wherein the trigger is selectively actuatable to actuate the end effector to thereby clamp tissue.

19. The surgical instrument of claim 12, wherein the fixed handle of the handle portion includes a battery compartment formed therein.

20. The surgical instrument of claim 19, wherein the battery compartment includes a door positioned at a bottom region of the fixed handle.
